(19) United States
(12) Patent Application Publication
(10) Pub. No.: US 2010/0130813 A1
(43) Pub. Date: May 27, 2010

(54) METHOD AND APPARATUS FOR ATTAINING LUCID DREAM STATE

(76) Inventor: Dmitri Dozortsev, Katy, TX (US)

Correspondence Address: DMITRI DOZORTSEV, 858 SHILLINGTON DR, KATY, TX, 77450, US

(21) Appl. No.: 12/623,400

(22) Filed: Nov. 21, 2009

Related U.S. Application Data
(60) Provisional application No. 61/117,424, filed on Nov. 24, 2008.

(51) Int. Cl. A61M 21/00

(52) U.S. Cl. 600/27

(57) ABSTRACT

The invention discloses an apparatus that facilitates attaining of lucid dreaming state.

This application claims priority of U.S. Provisional Patent Application Ser. No. 61/117424 filed Nov. 24, 2008, which is incorporated herein by reference.

### TECHNICAL FIELD OF INVENTION

The present invention is in the field of apparatuses for modulation of sleep pattern.

### BACKGROUND OF THE INVENTION

It is also known that some people may experience so-called lucid dreams during REM where the person is aware that he or she is dreaming while the dream is in progress. During lucid dreams, it is possible to exert conscious control over the dream characters and environment and have them perform feats which would be physically impossible in the waking world. Lucid dreams can be extremely real and vivid depending on a person's level of self-awareness during the lucid dream.

The number of people who are naturally predisposed to lucid dreaming is small.

However, there are methods that allow a not predisposed individual to experience lucid dreaming.

For example, it is well known that during night sleep humans have several episodes of dreaming accompanied by rapid eye movements (REM).

The devices for assisting lucid dreaming usually detect REM phase by monitoring eye movement using detectors mounted within the mask and then use flashing light or sound to let person know that he is dreaming without waking him up completely.

It has also been known for a long time that most men and woman experience respectively penis or clitoris erections and changes in blood flow and heart rate during the REM phase.

### SUMMARY OF THE INVENTION

The disclosed invention is designed to assist an individual to enter lucid dreaming filled with sexual experience, by providing sexual stimulation when REM is detected.

### DETAILED DESCRIPTION OF THE INVENTION

The disclosed apparatus has two principal parts, one detecting sexual arousal during sleep and second that provides a sexually stimulating stimulus once such arousal is detected. Because sexual arousal during sleep coincides with dreaming phase, it becomes possible for a user to introduce a sexual connotation into the dream.

Furthermore, recognizing external sexual stimulus, exerted by the apparatus, user will become aware of being dreaming, thereby transforming normal dream into lucid with sexual content.

Alternatively, the monitoring unit of disclosed apparatus may detect dream state by other means, for example by monitoring REMs or heart rate or blood pressure, skin resistance etc and send the signal to the second unit that provides sexual stimulation.

In a more simplified form, the arousal is not being monitored or detected. Instead, sexual stimulation is applied at predetermined programmed intervals during sleep.

### BRIEF DESCRIPTION OF THE DRAWING

FIG. 1 shows one of the possible realizations of the apparatus. In the shown variation, arousal detection and stimulation units are co-localized within the same ring, that can be positioned at the base of glance. A—with single unit, B—2 units.

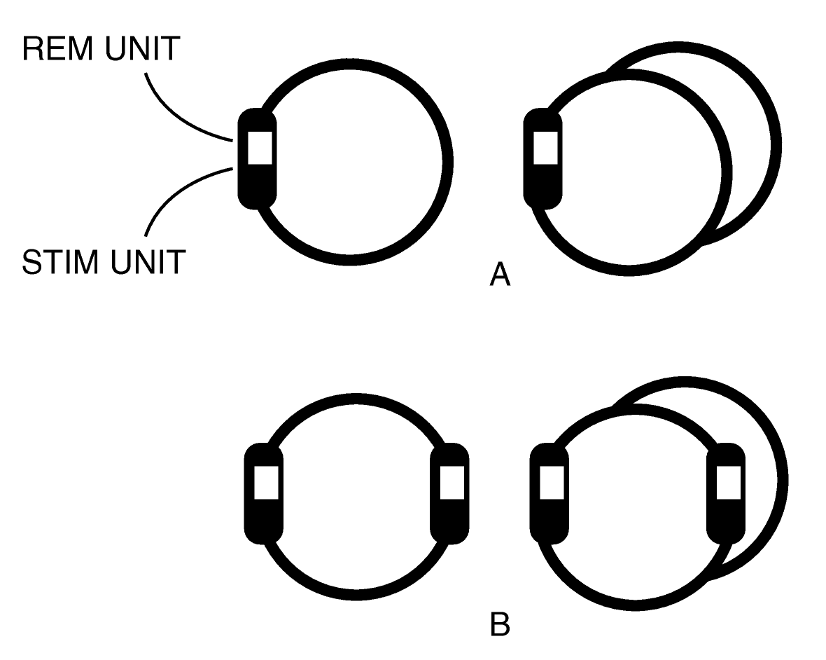

What claimed is:

1. The device that has a unit detecting REM stage (REM UNIT) and the unit inducing stimulation (STIM UNIT) once REM is detected.

2. The method of claim 1, where REM UNIT and STIM UNIT are communicating wirelessly

3. The method of claim 1, where REM UNIT and STIM UNIT are hardwired to each other

4. The method of claim 1, where REM UNIT detects REM stage by monitoring penile erection

5. The method of claim 4, where penile enlargement is detected

6. The method of claim 4, where changes in penile blood flow and/or heart rate are detected

7. The method of claim 1, where REM UNIT detects REM by monitoring clitoral erection

8. The method of claim 1, where REM UNIT detects REM by monitoring vaginal and clitoral blood flow.

9. The method of claim 1, where REM UNIT detects REM by monitoring eyes movements

10. The method of claim 1, where REM UNIT detects REM by monitoring peripheral blood flow

11. The method of claim 1, where REM UNIT detects REM by monitoring heart rate

12. The method of claim 1, where STIM UNIT is located on the penis, vagina and/or clitoris and/or breasts and/or in any number of erogenous areas of the male or female body.

13. The method of claim 1, where both, REM UNIT and STIM UNIT, are located on the penis, vagina and/or clitoris and/or breasts and/or in any number of erogenous areas of the male or female body.

14. The method of claim 1, where the device can be programmed to begin detecting REM stage after a predefined time.

15. The method of claim 1, where power of stimulation can be predefined or programmed

16. The method of claim 1, where the duration of stimulation can be predefined or programmed

17. The method of claim 1, where there are multiple REM UNITS and STIM UNITS

18. The method of claim 17, where multiple units are located in the same area of the body

19. The method of claim 17, where multiple units are located in different parts of the body

20. The method of claim 1, where STIM UNIT vibrates, rotates or interacts with adjusted tissues in some other manner.

21. The device that has only stimulation unit that provides sexual stimulation at predetermined, programmed intervals during sleep without detecting arousal or REM.
